(19) United States
(12) Patent Application Publication
(10) Pub. No.: US 2014/0277310 A1
(43) Pub. Date: Sep. 18, 2014

(54) Catheters Having Tethered Neuromodulation Units and Associated Devices, Systems, and Methods

(71) Applicant: MEDTRONIC ARDIAN LUXEMBOURG S.A.R.I., Luxembourg (LU)

(72) Inventors: Robert J. Beetel, Mountain View, CA (US); Maria G. Aboytes, Mountain View, CA (US); Mark S. Leung, Shawnigan Lake (CA)

(21) Appl. No.: 13/836,309

(22) Filed: Mar. 15, 2013

(51) Int. Cl. A61N 1/05

(52) U.S. Cl. CPC A61N 1/0551
USPC 607/116

(57) ABSTRACT

A catheter including an elongate shaft having a distal end portion locatable within or otherwise proximate to a body lumen of a patient, the catheter having a delivery state and a deployed state, and a tether secured to the neuromodulation unit and operationally associated with the shaft. The neuromodulation unit includes a therapeutic element and a support structure carrying the therapeutic element. The support structure is configured to resiliently urge the therapeutic element radially outward relative to a longitudinal axis of the support structure. The tether is sufficiently flexible to allow the neuromodulation unit to move independently of the distal end portion of the shaft when the catheter is in the deployed state and the neuromodulation unit is within the body lumen.

### TECHNICAL FIELD

The present technology is related to catheters. In particular, at least some embodiments are related to catheters including tethers configured to extend between neuromodulation units and distal end portions of associated shafts.

### BACKGROUND

The sympathetic nervous system (SNS) is a primarily involuntary bodily control system typically associated with stress responses. Fibers of the SNS extend through tissue in almost every organ system of the human body and can affect characteristics such as pupil diameter, gut motility, and urinary output. Such regulation can have adaptive utility in maintaining homeostasis or in preparing the body for rapid response to environmental factors. Chronic activation of the SNS, however, is a common maladaptive response that can drive the progression of many disease states. Excessive activation of the renal SNS in particular has been identified experimentally and in humans as a likely contributor to the complex pathophysiology of hypertension, states of volume overload (e.g., heart failure), and progressive renal disease.

Sympathetic nerves of the kidneys terminate in the renal blood vessels, the juxtaglomerular apparatus, and the renal tubules, among other structures. Stimulation of the renal sympathetic nerves can cause, for example, increased renin release, increased sodium reabsorption, and reduced renal blood flow. These and other neural-regulated components of renal function are considerably stimulated in disease states characterized by heightened sympathetic tone. For example, reduced renal blood flow and glomerular filtration rate as a result of renal sympathetic efferent stimulation is likely a cornerstone of the loss of renal function in cardio-renal syndrome, (i.e., renal dysfunction as a progressive complication of chronic heart failure). Pharmacologic strategies to thwart the consequences of renal sympathetic stimulation include centrally-acting sympatholytic drugs, beta blockers (e.g., to reduce renin release), angiotensin-converting enzyme inhibitors and receptor blockers (e.g., to block the action of angiotensin II and aldosterone activation consequent to renin release), and diuretics (e.g., to counter the renal sympathetic mediated sodium and water retention). These pharmacologic strategies, however, have significant limitations including limited efficacy, compliance issues, side effects, and others.

### BRIEF DESCRIPTION OF THE DRAWINGS

Many aspects of the present technology can be better understood with reference to the following drawings. The components in the drawings are not necessarily to scale. Instead, emphasis is placed on illustrating clearly the principles of the present technology. For ease of reference, throughout this disclosure identical reference numbers may be used to identify identical or at least generally similar or analogous components or features.

FIG. 1 is an anatomical side view illustrating a catheter and a sheath configured in accordance with an embodiment of the present technology; the catheter including a shaft and a neuromodulation unit. The catheter is shown in an intermediate state within a renal artery.

FIG. 2 is an anatomical side view illustrating the catheter and sheath shown in FIG. 1; the catheter further including a tether extending between a distal end portion of the shaft and the neuromodulation unit. The catheter is shown in a deployed state within the renal artery.

FIG. 3 is an enlarged, partially cross-sectional side view illustrating a junction between the distal end portion of the shaft and the neuromodulation unit shown in FIG. 1.

FIG. 4 is a cross-sectional end view of the junction shown in FIG. 3 with reference to the line 4-4 in FIG. 3.

FIG. 5 is a partially cross-sectional side view illustrating a junction between a distal end portion of a shaft and a neuromodulation unit of a catheter configured in accordance with another embodiment of the present technology.

FIG. 6 is an enlarged, conceptual side view illustrating interaction between locking features associated with the neuromodulation unit and locking features and notches associated with the distal end portion of the shaft within the junction shown in FIG. 5.

FIG. 7 is a partially cross-sectional, perspective side view illustrating a catheter configured in accordance with another embodiment of the present technology; the catheter including a shaft and a neuromodulation unit. The catheter is shown in a delivery state within a sheath.

FIG. 8 is a partially cross-sectional, perspective side view illustrating the catheter shown in FIG. 7 with the catheter shown in a deployed state.

FIG. 9 is a partially cross-sectional, perspective side view illustrating a catheter configured in accordance with another embodiment of the present technology; the catheter including a shaft and a neuromodulation unit. The catheter is shown in a deployed state.

FIG. 10 is a partially schematic illustration of a therapeutic system configured in accordance with an embodiment of the present technology; the system including the catheter shown in FIG. 7.

### DETAILED DESCRIPTION

Specific details of several embodiments of the present technology are described herein with reference to FIGS. 1-10. Although many of the embodiments are described herein with respect to devices, systems, and methods for intravascular renal neuromodulation, other applications and other embodiments in addition to those described herein are within the scope of the present technology. For example, at least some embodiments may be useful for intraluminal neuromodulation, for extravascular neuromodulation, for non-renal neuromodulation, and/or for use in therapies other than neuromodulation. It should be noted that other embodiments in addition to those disclosed herein are within the scope of the present technology. For example, embodiments of the present technology can have different configurations, components, and/or procedures than those shown or described herein. Moreover, a person of ordinary skill in the art will understand that embodiments of the present technology can have configurations, components, and/or procedures in addition to those shown or described herein and that these and other embodiments can be without several of the configurations, components, and/or procedures shown or described herein without deviating from the present technology.

As used herein, the terms “distal” and “proximal” define a position or direction with respect to a clinician or a clinician's control device (e.g., a handle of a catheter). The terms “distal” and “distally” refer to a position distant from or in a direction away from a clinician or a clinician's control device. The terms “proximal” and “proximally” refer to a position near or in a direction toward a clinician or a clinician's control device. The headings provided herein are for convenience only and should not be construed as limiting the subject matter disclosed.

It is typically advantageous to at least generally maintain the position of a neuromodulation unit relative to the surrounding anatomy during a neuromodulation treatment. For example, it can be advantageous to at least generally maintain stable contact between a therapeutic element of a neuromodulation unit and an inner wall of a body lumen (e.g., a blood vessel, a duct, an airway, or another naturally occurring lumen within the human body) during a neuromodulation treatment. This can enhance control and/or monitoring of the treatment, reduce trauma to the body lumen, and/or have other advantages. In some cases, at least generally maintaining the position of a neuromodulation unit relative to the surrounding anatomy during a neuromodulation treatment can be challenging. For example, a patient's adjacent body tissues may move (e.g., in response to respiration or cardiac pulsation) and/or a shaft connected to a neuromodulation unit may move (e.g., in response to a handle connected to the shaft being inadvertently bumped, jostled, or otherwise disturbed) during a neuromodulation treatment. Such movement of a patient's body and/or a shaft can cause disadvantageous relative movement between a neuromodulation unit connected to the shaft and the surrounding anatomy at a target site.

Another problem may exist with respect to initial positioning of a neuromodulation unit. When a neuromodulation unit is initially positioned at a treatment location within a body lumen (e.g., within a renal artery) the position of the neuromodulation unit may be suboptimal. For example, a catheter and/or a sheath carrying the catheter may be insufficiently flexible to match the curvature of anatomy near the treatment location (e.g., the curvature of a renal ostium between a renal artery and an aorta). This may cause the catheter and/or the sheath to enter the body lumen out of alignment with the body lumen (e.g., out of alignment with a longitudinal axis of the body lumen). When a neuromodulation unit of a misaligned catheter is initially moved into an expanded form, the neuromodulation unit may also not be aligned with the body lumen. Such misalignment of a neuromodulation unit may also occur for other reasons. Misalignment of a neuromodulation unit can be problematic. For example, when a neuromodulation unit is misaligned, one or more therapeutic elements of the neuromodulation unit may be out of contact or in poor contact with an inner wall of a body lumen. Even when the neuromodulation unit is sufficiently well aligned for treatment to begin, misalignment may occur later, disturbing the wall contact and requiring the treatment to be aborted. Correcting misalignment of a neuromodulation unit can be challenging when the neuromodulation unit remains directly attached to an associated shaft.

Catheters configured in accordance with at least some embodiments of the present technology can at least partially address one or more of the problems described above and/or other problems associated with conventional technologies whether or not stated herein. For example, a catheter configured in accordance with a particular embodiment of the present technology includes a neuromodulation unit configured to move freely with the surrounding anatomy. In some embodiments, a catheter includes a neuromodulation unit tethered to a shaft such that the neuromodulation unit can move more freely relative to the shaft than if the neuromodulation unit were directly attached to the shaft. This can reduce or prevent disadvantageous relative movement between the neuromodulation unit and the surrounding anatomy. Furthermore, this can facilitate repositioning the neuromodulation unit when the neuromodulation unit is misaligned with a body lumen.

### Selected Examples of Catheters and Related Devices

FIGS. 1 and 2 are anatomical side views illustrating a catheter 100 and a sheath 102 configured in accordance with an embodiment of the present technology. In FIGS. 1 and 2, the catheter 100 is shown in an intermediate state and a deployed state, respectively, within a renal artery 104 of a human patient. In a delivery state, catheter 100 would appear similar to catheter 700 shown in FIG. 7. With reference to FIGS. 1 and 2 together, the catheter 100 can include an elongate shaft 106 and a neuromodulation unit 108 operably connected to the shaft 106. The shaft 106 can include a distal end portion 110, and the shaft 106 can be configured to locate the distal end portion 110 within or otherwise proximate to a body lumen (e.g., the renal artery 104 or another suitable body lumen). The neuromodulation unit 108 can be operably connected to the shaft 106 via the distal end portion 110 and can have a suitable location within the body lumen when the distal end portion 110 is located within or otherwise proximate to the body lumen. When the catheter 100 is in a delivery state within the sheath 102, the shaft 106 and the neuromodulation unit 108 can be 2, 3, 4, 5, 6, or 7 French or other suitable sizes.

The neuromodulation unit 108 can have a low-profile collapsed form (not shown) in which the neuromodulation unit 108 is radially or transversely constrained within the sheath 102. The neuromodulation unit 108 can also have an expanded form (as illustrated in FIGS. 1 and 2) when the catheter 100 is in the deployed and intermediate states. Among other suitable expanded forms, the neuromodulation unit 108 can have a helical expanded form (e.g., a coiled, spiral, or other similar form having two or more turns consistently or variably spaced along a longitudinal axis of the neuromodulation unit 108 and having a consistent or variable transverse dimension along the longitudinal axis) when the catheter 100 is in the deployed and intermediate states.

In some embodiments, intravascular delivery of the catheter 100 includes percutaneously inserting a guide wire (not shown) into a body lumen of a patient, and moving the shaft 106 and the neuromodulation unit 108 along the guide wire until the neuromodulation unit 108 reaches a suitable treatment location (e.g., within the renal artery 104). In other embodiments, the catheter 100 can be a steerable or non-steerable device configured for use without a guide wire. In still other embodiments, the catheter 100 can be configured to be positioned via the sheath 102, which can be pre-curved, steerable and/or configured for intravascular delivery via a guide wire.

The neuromodulation unit 108 can be configured to be directly attached to the distal end portion 110 at some times during use of the catheter 100 and separated (e.g., decoupled) from the distal end portion 110 at other times during use of the catheter 100. For example, the neuromodulation unit 108 can be directly attached to the distal end portion 110 by a mechanical junction 115 configured to allow the neuromodulation unit 108 to separate from the distal end portion 110. In FIG. 1, the neuromodulation unit 108 is shown in its expanded form within the renal artery 104 and directly attached to the distal end portion 110 while the catheter 100 is in the intermediate state. In FIG. 2, the neuromodulation unit 108 is shown in its expanded form within the renal artery 104 and is separated from the distal end portion 110 while the catheter 100 is in the deployed state. In some embodiments, the catheter 100 is configured to transform the neuromodulation unit 108 from the collapsed form to the expanded form as the sheath 102 is retracted proximally relative to the catheter 100 and/or as the catheter 100 is advanced distally relative to the sheath 102. The neuromodulation unit 108 can be in the expanded form and still attached to the distal end portion 110 when the catheter 100 reaches the intermediate state. From the intermediate state, the neuromodulation unit 108 can remain in the expanded form and then be separated from the distal end portion 110 to transform the catheter 100 into the deployed state. In other embodiments, the catheter 100 can be configured to transform from the delivery state directly to the deployed state without an intervening intermediate state. For example, the sheath 102 can be retracted proximally relative to the catheter 100 and/or the catheter 100 can be advanced distally relative to the sheath 102 to cause the neuromodulation unit 108 to simultaneously separate from the distal end portion 110 and expand into the expanded form.

The neuromodulation unit 108 can be configured to modulate one or more nerves within tissue at or otherwise proximate to a wall of a body lumen. For example, the neuromodulation unit 108 can include a support structure 112 and one or more therapeutic elements 114 coupled to the support structure 112 configured for delivering energy to, withdrawing energy from, delivering a chemical to, or otherwise interacting with tissue during a neuromodulation treatment. Accordingly, the therapeutic elements 114 can include electrodes, cryotherapeutic applicators, direct-heat applicators, ultrasound transducers, chemical ports, optical elements for delivering laser light, light emitting diodes, or other suitable structures configured to interact with tissue during a neuromodulation treatment. In some embodiments, the support structure 112 is configured to resiliently expand in a radial or transverse direction when the sheath 102 is retracted proximally relative to the catheter 100 and/or the catheter 100 is advanced distally relative to the sheath 102. In other embodiments, the support structure 112 can be configured to expand in another suitable manner or be non-expanding. When the neuromodulation unit 108 is in the expanded form, the support structure 112 can resiliently urge the therapeutic elements 114 transverse to a longitudinal axis of the support structure 112. This can facilitate operable engagement between the therapeutic elements 114 and the inner wall of the body lumen over a broad range of anatomical variation.

In some embodiments, the catheter 100 is configured to maintain at least generally stable contact between the therapeutic elements 114 and an inner wall of a body lumen during a neuromodulation treatment. The catheter 100 can include a tether 116 (FIG. 2) with one end secured to the neuromodulation unit 108 and another end operationally associated with (e.g., secured to and/or extending through) the shaft distal end portion 110. In some embodiments, the tether 116 is made at least partially of a polymer (e.g., a fluoropolymer (e.g., polytetrafluoroethylene)), a para-aramid, or another suitable polymer). In other embodiments, the tether 116 can have other suitable compositions. The tether 116 can be sufficiently flexible to allow the neuromodulation unit 108 to move independently of the distal end portion 110 (e.g., in response to a patient's respiration or cardiac pulsation) when the catheter 100 is in the deployed state. For example, the tether 116 can be a flexible cord, line, or other suitable elongate member configured to bend and flex relatively easily in response to differential movement of the neuromodulation unit 108 and the distal end portion 110. The tether 116 can flexibly accommodate this differential movement, thereby decreasing the probability of dislodging the therapeutic elements 114 from the inner wall of a body lumen during a neuromodulation treatment. In some cases, the tether 116 accommodates this differential movement while the neuromodulation unit 108 is within the renal artery 104, the distal end portion 110 is within an aorta 118 of the patient, and the tether 116 extends through a renal ostium 120 of the patient. In other cases, the tether 116 accommodates this differential movement from another suitable anatomical position.

The tether 116 can be configured to restrict a longitudinal separation distance between the neuromodulation unit 108 and the distal end portion 110. This can be useful, for example, to reduce or prevent the neuromodulation unit 108 from being carried away (e.g., by flowing blood) if it is dislodged from a treatment location within a body lumen. In some embodiments, a maximum longitudinal extension of the tether 116 is selected based on anatomical structures in the vicinity of a treatment location. For example, when the neuromodulation unit 108 is configured to be positioned within the renal artery 104 and the distal end portion 110 is configured to be positioned within the aorta 118, the tether 116 can be have a maximum longitudinal extension sufficient to allow the tether 116 to extend through the renal ostium 120.

In some cases, the tether 116 provides one or more advantages in addition to or instead of accommodating differential movement of the neuromodulation unit 108 and the distal end portion 110. For example, as discussed above, when the neuromodulation unit 108 is initially moved into its expanded form within a body lumen, the position of the neuromodulation unit 108 may be suboptimal. The tether 116 can reposition (e.g., alignment, centering, or other types of repositioning) the neuromodulation unit 108 while the catheter 100 is in the deployed state by applying tension on the neuromodulation unit 108 from the distal end portion 110. In a particular embodiment, the neuromodulation unit 108 is configured to at least partially self-align within the renal artery 104 by moving proximally along a longitudinal axis of the renal artery 104 in response to tension on the tether 116 when the catheter 100 is in the deployed state. For example, when the neuromodulation unit 108 is in the expanded form to resiliently urge the therapeutic elements 114 radially outward relative to a longitudinal axis of the neuromodulation unit 108, pulling the neuromodulation unit 108 in a direction, e.g. proximally, at least generally aligned with a longitudinal axis of the renal artery 104 can cause the support structure 112 to tend to evenly redistribute the radially directed resilient force, thereby causing the neuromodulation unit 108 to move toward better alignment with the longitudinal axis of the renal artery 104 without disengaging the inner wall of the renal artery 104.

Using the tether 116, the neuromodulation unit 108 can be pulled in one or more at least generally proximal directions, such as by moving the distal end portion 110 to one or more different positions within the aorta 118 and pulling the tether 116 taut. When the tether 116 is taut, the anatomical location of a distalmost contact point between the tether 116 and the distal end portion 110 can determine a direction in which the neuromodulation unit 108 is pulled proximally within the renal artery. The distalmost contact point, for example, can be at an attachment point between the tether 116 and the distal end portion 110, a point at which the tether 116 bends around a distal rim 122 of the distal end portion 110, or another suitable point. In a particular example, a distalmost contact point between the tether 116 and the distal end portion 110 is positioned within the aorta 118 and is at least generally aligned with a longitudinal axis of the renal artery 104, and tension of the tether 116 pulls the neuromodulation unit 108 in a direction at least generally aligned with the longitudinal axis of the renal artery 104. In some embodiments, the tether 116 is fixedly attached to the distal end portion 110. For example, the tether 116 can be secured to the distal end portion 110 (not shown) and the amount of slack in or tension on the tether 116 can be solely a function of the longitudinal separation distance between the neuromodulation unit 108 and the distal end portion 110 and not subject to independent operator control. In other embodiments, the tether 116 can extend through the distal end portion 110. For example, the tether 116 can extend to a handle of the catheter 100 and the handle can be configured with an actuator to control the amount of slack in or tension on the tether 116. In catheter 100, these features can be similar to handle 1006 and actuator 1109 illustrated in FIG. 10 and described in detail below.

FIG. 3 is an enlarged, partially cross-sectional side view illustrating the junction 115 between the distal end portion 110 of the shaft 106 and the neuromodulation unit 108. The junction 115 is shown in a closed (e.g., coupled) state such that the catheter 100 is in a delivery state or an intermediate state. FIG. 4 is a cross-sectional end view of the junction 115 with reference to the line 4-4 in FIG. 3. With reference to FIGS. 1-4 together, the neuromodulation unit 108 can include a proximal hub 124 secured to the tether 116 (e.g., at a knot 125 or another suitable attachment point of the proximal hub 124), and the distal end portion 110 can include a bore 126 configured to receive at least a portion of the proximal hub 124. For example, the junction 115 can be a mating junction, the proximal hub 124 can include a proximal pin 128, and the proximal pin 128 can be configured to move into the bore 126 in response to tension on the tether 116. In some embodiments, the tether 116 is secured to and/or extends through the proximal pin 128 and at least a portion of the proximal pin 128 is tapered with decreasing width in a direction away from the support structure 112. These features can guide the proximal pin 128 into the bore 126 and/or have other benefits. In other embodiments, the proximal pin 128 can be non-tapered, rounded, or have another suitable form.

As shown in FIG. 4, the tether 116 can include an electrical lead 130. The electrical lead 130 can be flexible and the tether 116 can be configured to carry the electrical lead 130 between the distal end portion 110 and the neuromodulation unit 108 when the catheter 100 is in the deployed state. The electrical lead 130 can be operably connected to one or more of the therapeutic elements 114, such as to supply one or more of the therapeutic elements 114 with power during a neuromodulation treatment. Although only one electrical lead 130 is shown in FIG. 4, in other embodiments the tether 116 can carry a plurality of independently operable electrical leads 130 (e.g., an electrical lead 130 corresponding to each of the therapeutic elements 114), one or more sensor leads (e.g., thermocouple leads), and/or other suitable lines (e.g., leads for electrical grounding or tubular lines for refrigerant supply, refrigerant venting, chemical supply, or other suitable purposes). When the tether 116 carries a plurality of independently operable electrical leads 130, the catheter 100 can be operably connected to a multi-channel generator for independent operation of the therapeutic elements 114.

When the catheter 100 is in the delivery state or the intermediate state, the neuromodulation unit 108 and the distal end portion 110 are translationally coupled such that the junction 115 is in a closed, coupled or mated state. When the catheter 100 is in the deployed state, the proximal hub 124 and the distal end portion 110 are translationally decoupled such that the junction 115 is in an open or decoupled state. When translationally coupled, one or more of distal longitudinal movement of the distal end portion 110, proximal longitudinal movement of the distal end portion 110, and rotation of the distal end portion 110 about an axis of the distal end portion 110 can be translated to the neuromodulation unit 108. When translationally decoupled, the neuromodulation unit 108 can be independent of these types of movement of the distal end portion 110. In some embodiments, the neuromodulation unit 108 is rotationally interlocked with the distal end portion 110 when the catheter 100 is in the delivery state. For example, a proximal rim 132 of the proximal hub 124 and the distal rim 122 of the distal end portion 110 can include sets of teeth 134 configured to mate or interlock under force applied by tension in tether 166 when the catheter 100 is in the delivery state. Mating sets of teeth 134 are shown disengaged in FIG. 2. In other embodiments, the neuromodulation unit 108 can be independent of rotation of the shaft distal end portion 110 about its axis when the catheter 100 is in the delivery state. Rotationally interlocking the neuromodulation unit 108 and the distal end portion 110 when the catheter 100 is in the delivery state can be useful, for example, to facilitate control over a circumferential position of the neuromodulation unit 108 relative to the distal end portion 110 during delivery of the neuromodulation unit 108, to reduce or prevent twisting of the tether 116 during delivery of the neuromodulation unit 108, and/or for other reasons.

To summarize some of the functions of tether 116 and junction 115 as exemplified in catheter 100, neuromodulation unit 108 is always connected (e.g. in the catheter delivery state, intermediate state, or deployed state) via tether 116 to either catheter shaft distal end portion 110, catheter shaft 106, or to handle 1006. Tether 116 contains one or more electrical leads and/or tubular lines to carry different forms of energy to and from neuromodulation unit 108 to perform neuromodulation via the corresponding modality. Neuromodulation unit 108 is also mated via junction 115 to catheter shaft distal end portion 110, but only in the catheter delivery state or the intermediate state. Other embodiments of the present technology, including tethers 505 and 707 function similarly.

FIG. 5 is a partially cross-sectional side view illustrating a junction 500 between a distal end portion 502 of the shaft 106 and a neuromodulation unit 504 of a catheter configured in accordance with another embodiment of the present technology. The junction 500 is shown in an open (e.g., decoupled) state with a tether 505 extending between the distal end portion 502 and a neuromodulation unit 504 such that the catheter is in a deployed state. The neuromodulation unit 504 can include a proximal hub 506 having a proximal pin 508 with an outer surface 509. The proximal pin 508 can include two or more first locking features 510 circumferentially spaced apart around the outer surface 509 and two or more first intervening regions 512 individually positioned between circumferentially adjacent first locking features 510. The individual first locking features 510 can include a first distal edge portion 510a and a first proximal edge portion 510b. The distal end portion 502 can include a bore 514 having an inner surface 515. The bore 514 can include two or more second locking features 518 circumferentially spaced apart around the inner surface 515 and two or more second intervening regions 519 individually positioned between circumferentially adjacent second locking features 518. The second locking features 518 can be configured to interface with the first locking features 510 to releasably couple the proximal hub 506 of the neuromodulation unit 504 to the bore 514 of the distal end portion 502. For example, the individual second locking features 518 can include a second distal edge portion 518a and a second proximal edge portion 518b. The individual second proximal edge portions 518b can include one or more first notches 520. The bore 514 can further include a ledge 522 having two or more second notches 524. The tether 505 can include a first stop 516 and the distal end portion 502 can include a second stop 517 configured to engage the first stop 516 to restrict a separation distance between the distal end portion 502 and the neuromodulation unit 504 when the catheter is in the deployed state.

FIG. 6 is an enlarged, conceptual side view illustrating interaction between the first locking features 510 and the second locking features 518, the first notches 520, and the second notches 524. The proximal pin 508 can be configured to move into the bore 514 in response to a first period of tension on the tether 505. For example, the first locking features 510 can be configured to move longitudinally through the second intervening regions 519 and the first locking features 510 can be configured to move longitudinally through the first intervening regions 512 as the proximal pin 508 moves into the bore 514 in response to the first period of tension on the tether 505. After the first period of tension of the tether 505, the junction 500 can be configured to remain closed until a second period of tension of the tether 505 temporally spaced apart from and following the first period. The second period of tension of the tether 505 can be used to open the junction 500 (e.g., so as to allow the catheter to transform into the deployed state).

The distal end portion 502 can include a resilient member 526 (e.g., a coil spring) operably positioned within the bore 514 and configured to resiliently deform as the proximal pin 508 moves into the bore 514 in response to the first period of tension on the tether 505. As the proximal pin 508 moves into the bore 514, the first proximal edge portions 510b can interact with the second distal edge portions 518a to cause the proximal pin 508 to rotate relative to the bore 514 so as to move the first locking features 510 into alignment with the second intervening regions 519. As the proximal pin 508 moves further into the bore 514 (arrow 528), the proximal edge portions 510b can interact with the second notches 524 to cause the proximal pin 508 to rotate relative to the bore 514 so as to move the first locking features 510 from being aligned with the second intervening regions 519 toward being out of alignment with the second intervening regions 519. The first period of tension of the tether 505 can then be released to cause the resilient member 526 to resiliently urge the first locking features 510 into operable engagement with the second locking features 518 (arrow 530). For example, the first distal edge portions 510a can be configured to interlock with the first notches 520 as the resilient member 526 urges the first locking features 510 into operable engagement with the second locking features 518. The second period of tension on the tether 505 can overcome force from the resilient member 526 and move the proximal pin 508 proximally (arrow 532). As the proximal pin 508 moves proximally in response to the second period of tension on the tether 505 (arrow 532), the first proximal edge portions 510b can interact with the second notches 524 to cause the proximal pin 508 to rotate relative to the bore 514 so as to partially move the first locking features 510 from being out of alignment with the second intervening regions 519 toward being aligned with the second intervening regions 519. The second period of tension on the tether 505 can then be released such that the resilient member 526 moves the proximal pin 508 distally outward (arrow 534). As the proximal pin 508 moves distally outward (arrow 534), the first distal edge portions 510a can interact with the second proximal edge portions 518b to cause the proximal pin 508 to rotate relative to the bore 514 so as to further move the first locking features 510 from being out of alignment with the second intervening regions 519 toward being aligned with the second intervening regions 519.

FIGS. 7 and 8 are partially cross-sectional, perspective side views illustrating a catheter 700 within a sheath 701 in accordance with another embodiment of the present technology. The catheter 700 can include an elongate shaft 702 and a neuromodulation unit 704 operably connected to the shaft 702 by tether 707. The neuromodulation unit 704 can also include a proximal hub 708 that mates with a distal rim 710 of the shaft distal end portion 706 to form a junction therebetween when the catheter 700 is in the delivery state (FIG. 7) or in an intermediate state (not shown, but see FIG. 1, which is similar).

The neuromodulation unit 704 can include first and second arms 712, 714 extending distally from the proximal hub 708. The first arm 712 can have a distal tip 716 and can carry a first therapeutic element 718 at least proximate to the distal tip 716. Similarly, the second arm 714 can have a distal tip 720 and can carry a second therapeutic element 722 at least proximate to the distal tip 720. The first and second arms 712, 714 can be pre-formed to resiliently splay such that distal tips 716, 720 spread apart in opposite directions transverse to the longitudinal axis of the neuromodulation unit 704 while the proximal ends of the arms 712, 714 remain fixed at the proximal hub 708. Other embodiments may have more than two arms carrying therapeutic elements.

In the embodiment shown in FIG. 7, with the catheter in the delivery state, the proximal hub 708 abuts the distal rim 710 and the proximal end of tether 707 is affixed inside catheter shaft 702 such that the slack tether 707 is loosely stored within the distal end portion 706. Sheath 701 constrains the first and second arms 712, 714 from their natural tendency to splay. The catheter 700 is transformed from the delivery state to the intermediate state by moving the catheter 700 distally relative to the sheath 701 and/or moving the sheath 701 proximally relative to the catheter 700. During this transformation, the neuromodulation unit 704 is exposed from sheath 701, thereby releasing first and second arms 712, 714 to expand toward their pre-formed shape, preferably at a desired target location within the vessel, similar to the illustration in FIG. 1. During this transformation, tether 707 remains slack inside distal end 706 while the proximal hub 708 maintains contact with the distal rim 710 either by the distal advancement of shaft 702 against the neuromodulation unit 704 or by the neuromodulation unit 704 being drawn proximally against shaft 702 by sheath 701. Next, The catheter 700 is transformed from the intermediate state to the deployed state by moving the catheter 700 proximally to separate the distal rim 710 from the proximal hub 708, thereby permitting the neuromodulation unit 704 to seek a stable aligned position with the vessel, fettered only by flexible tether 707 (See FIGS. 2 and 8).

To remove the neuromodulation unit 704 from the targeted vessel location (e.g. after a neuromodulation treatment), the tether 707 can be drawn taut to act as a guide for distal advancement of sheath 701 over the neuromodulation unit 704 with or without re-coupling the distal rim 710 and the proximal hub 708. Such re-coupling can be accomplished by passing the shaft 702 over the taut tether 707 until the catheter 700 is once again in the intermediate state. Alternatively, the catheter 700 can be withdrawn proximally into the sheath 701 without re-coupling the distal rim 710 and the proximal hub 708. In this method, withdrawing the catheter 700 applies tension to the tether 707 to pull the neuromodulation unit 704 into the sheath 701 without. In yet another method, the tether 707 can be withdrawn proximally into the shaft 702 to re-couple the distal rim 710 and the proximal hub 708 until the catheter 700 is once again in the intermediate state. Then, the catheter 700 can be withdrawn proximally into the sheath 701. In any of the methods of removing the neuromodulation unit 704 from the targeted vessel location, the sheath 701 again constrains the first and second arms 712, 714 from their natural tendency to splay. The sheath can then be removed from the patient or re-positioned to perform neuromodulation in a contralateral renal artery using the same catheter 700 or a different catheter. All of the methods of use described herein, including the transformation of the catheters between various states or configurations and the steps for withdrawal of the neuromodulation unit from the targeted vessel location are similar for catheters 100, 700 and 900.

The amount of slack in or tension on the tether 707 can be solely a function of the longitudinal separation distance between the neuromodulation unit 704 and the distal end portion 706 and not subject to independent operator control. For example, when the catheter 700 is in the deployed state, the tether 707 can extend longitudinally until taut, at which point the tether 707 can restrict the longitudinal separation distance between the neuromodulation unit 704 and the distal end portion 706. After the catheter 700 has been in the deployed state, the neuromodulation unit 704 and the distal end portion 706 can be moved back into the sheath 701 while the tether 707 remains longitudinally extended and taut (e.g., by moving the sheath 701 distally relative to the catheter 700 and/or by moving the catheter 700 proximally relative to the sheath 701). Within the sheath 701, the tether 707 can remain extended or move back into the distal end portion 706. For example, in some embodiments, the tether 707 can be resilient and configured to assume a compact form (e.g., a coiled form as illustrated in FIG. 7) within the distal end portion 706 when the neuromodulation unit 704 and the distal end portion 706 are not longitudinally spaced apart. This can reduce the possibility of tangling the tether 707 and/or can have other advantages. In other embodiments, the tether 707 can be non-resilient. In other embodiments (not shown), tether 707 can extend proximally through shaft 706 to handle 1006 where actuator 1009 can be operated to close the longitudinal separation distance between the neuromodulation unit 704 and the distal end portion 706.

FIG. 9 is a partially cross-sectional, perspective side view illustrating a catheter 900 configured in accordance with another embodiment of the present technology. The catheter 900 can include a neuromodulation unit 902 operably connected to the shaft 702. The neuromodulation unit 902 can include a proximal hub 904, a distal hub 906, and three arms (individually identified as 908a-c) extending therebetween. The individual arms 908a-c can include a distal portion (individually identified as 910a-c), a proximal portion (individually identified as 912a-c), and a therapeutic element (individually identified as 914a-c) therebetween. The therapeutic elements 914a-c can be configured to contact different segments of a renal artery inner wall. In some embodiments, the neuromodulation unit 902 can further include a central support 916 (e.g., a control rod) extending between the proximal and distal hubs 904, 906. The distal portions 910a-c of the arms 908a-c can be resilient and configured to splay when the catheter 900 is expelled from the sheath 701. The proximal portions 912a-c of the arms 908a-c may be flexible filaments that serve to guide the therapeutic elements 914a-c into the open distal end of distal end portion 706 when the neuromodulation unit 902 is withdrawn into the shaft 702 (e.g., in response to tension on the tether 707). The proximal hub 904 mates with the distal rim 710 of the shaft distal end portion 706 to form a junction therebetween when the catheter 900 is in the delivery state (not shown, but see FIG. 7, which is similar) or in an intermediate state (not shown, but see FIG. 1, which is similar). Other embodiments may have only two arms, or more than three arms carrying therapeutic elements.

### Selected Examples of Neuromodulation Systems

FIG. 10 is a partially schematic illustration of a therapeutic system 1000 configured in accordance with an embodiment of the present technology. The system 1000 can include any of the catheters 100 (FIGS. 1-6), 700 (FIGS. 7 and 8), 900 (FIG. 9), or another of the catheters described herein, a console 1002, and a cable 1004 extending therebetween. The catheter 100, 700, 900 can include a handle 1006 operably connected to the shaft 106, 702 via a proximal end portion 1008 of the shaft 106, 702. In some embodiments, the handle 1006 is configured to control the amount of slack in or tension on a tether (not shown) of the catheters 100, 700, 900. For example, the handle 1006 can include an actuator 1009 configured to transform a junction (not shown) between the distal end portion 110, 504, 706 of the shaft 106, 702 and the neuromodulation unit 108, 504, 704, 902 of the catheter 100 from a closed (e.g., coupled) state to an open (e.g., decoupled state), from the closed state to the open state, or both from the open state to the closed state and from the closed state to the open state. The actuator 1009 can be electronic, manual, or have another suitable modality. Furthermore, the actuator 1009 can include a reel (e.g., a motorized reel), a knob, a dial, a pin, a lever, or another suitable control component operably connected to the tether.

The console 1002 can be configured to control, monitor, supply, and/or otherwise support operation of the catheter 100, 700, 900. Alternatively, the catheter 100, 700, 900 can be self-contained or otherwise configured for operation without connection to the console 1002. When present, the console 1002 can be configured to generate a selected form and/or magnitude of energy for delivery to tissue at a treatment location via the neuromodulation unit 108, 504, 704, 902 (shown schematically in FIG. 10). The console 1002 can have different configurations depending on the treatment modality of the catheter 100, 700, 900. When the catheter 100, 700, 900 is configured for electrode-based, heat-element-based, or transducer-based treatment, for example, the console 1002 can include an energy generator (not shown) configured to generate radiofrequency energy, pulsed energy, microwave energy, optical energy, ultrasound energy (e.g., focused ultrasound energy, such as high-intensity focused ultrasound energy), thermal energy (e.g., direct heat), light, or another suitable type of energy. When the catheter 100, 700, 900 is configured for cryotherapeutic treatment, the console 1002 can include a refrigerant reservoir (not shown) and can be configured to supply the catheter 100, 700, 900 with refrigerant. Similarly, when the catheter 100, 700, 900 is configured for chemical-based treatment (e.g., drug infusion), the console 1002 can include a chemical reservoir (not shown) and can be configured to supply the catheter 100, 700, 900 with one or more chemicals.

In some embodiments, the system 1000 includes a control device 1010 along the cable 1004. The control device 1010 can be configured to initiate, terminate, and/or adjust operation of one or more components of the catheter 100, 700, 900 directly and/or via the console 1002. In other embodiments, the control device 1010 can be absent or have another suitable location (e.g., within the handle 1006). The console 1002 can be configured to execute an automated control algorithm 1012 and/or to receive control instructions from an operator. Furthermore, the console 1002 can be configured to provide feedback to an operator before, during, and/or after a treatment procedure via an evaluation/feedback algorithm 1014.

### Renal Neuromodulation

Renal neuromodulation is the partial or complete incapacitation or other effective disruption of nerves of the kidneys (e.g., nerves terminating in the kidneys or in structures closely associated with the kidneys). In particular, renal neuromodulation can include inhibiting, reducing, and/or blocking neural communication along neural fibers (e.g., efferent and/or afferent neural fibers) of the kidneys. Such incapacitation can be long-term (e.g., permanent or for periods of months, years, or decades) or short-term (e.g., for periods of minutes, hours, days, or weeks). Renal neuromodulation is expected to contribute to the systemic reduction of sympathetic tone or drive and/or to benefit at least some specific organs and/or other bodily structures innervated by sympathetic nerves. Accordingly, renal neuromodulation is expected to be useful in treating clinical conditions associated with systemic sympathetic overactivity or hyperactivity, particularly conditions associated with central sympathetic overstimulation. For example, renal neuromodulation is expected to efficaciously treat hypertension, heart failure, acute myocardial infarction, metabolic syndrome, insulin resistance, diabetes, left ventricular hypertrophy, chronic and end stage renal disease, inappropriate fluid retention in heart failure, cardio-renal syndrome, polycystic kidney disease, polycystic ovary syndrome, osteoporosis, erectile dysfunction, and sudden death, among other conditions.

Renal neuromodulation can be electrically-induced, thermally-induced, chemically-induced, or induced in another suitable manner or combination of manners at one or more suitable treatment locations during a treatment procedure. The treatment location can be within or otherwise proximate to a renal lumen (e.g., a renal artery, a ureter, a renal pelvis, a major renal calyx, a minor renal calyx, or another suitable structure), and the treated tissue can include tissue at least proximate to a wall of the renal lumen. For example, with regard to a renal artery, a treatment procedure can include modulating nerves in the renal plexus, which lay intimately within or adjacent to the adventitia of the renal artery.

Renal neuromodulation can include a cryotherapeutic treatment modality alone or in combination with another treatment modality. Cryotherapeutic treatment can include cooling tissue at a treatment location in a manner that modulates neural function. For example, sufficiently cooling at least a portion of a sympathetic renal nerve can slow or potentially block conduction of neural signals to produce a prolonged or permanent reduction in renal sympathetic activity. This effect can occur as a result of cryotherapeutic tissue damage, which can include, for example, direct cell injury (e.g., necrosis), vascular or luminal injury (e.g., starving cells from nutrients by damaging supplying blood vessels), and/or sublethal hypothermia with subsequent apoptosis. Exposure to cryotherapeutic cooling can cause acute cell death (e.g., immediately after exposure) and/or delayed cell death (e.g., during tissue thawing and subsequent hyperperfusion). Neuromodulation using a cryotherapeutic treatment in accordance with embodiments of the present technology can include cooling a structure proximate an inner surface of a vessel or chamber wall such that tissue is effectively cooled to a depth where sympathetic renal nerves reside. For example, in some embodiments, a cooling assembly of a cryotherapeutic device can be cooled to the extent that it causes therapeutically-effective, cryogenic renal neuromodulation. In other embodiments, a cryotherapeutic treatment modality can include cooling that is not configured to cause neuromodulation. For example, the cooling can be at or above cryogenic temperatures and can be used to control neuromodulation via another treatment modality (e.g., to protect tissue from neuromodulating energy).

Renal neuromodulation can include an electrode-based or transducer-based treatment modality alone or in combination with another treatment modality. Electrode-based or transducer-based treatment can include delivering electricity and/or another form of energy to tissue at a treatment location to stimulate and/or heat the tissue in a manner that modulates neural function. For example, sufficiently stimulating and/or heating at least a portion of a sympathetic renal nerve can slow or potentially block conduction of neural signals to produce a prolonged or permanent reduction in renal sympathetic activity. A variety of suitable types of energy can be used to stimulate and/or heat tissue at a treatment location. For example, neuromodulation in accordance with embodiments of the present technology can include delivering radiofrequency energy, pulsed energy, microwave energy, optical energy, focused ultrasound energy (e.g., high-intensity focused ultrasound energy), or another suitable type of energy alone or in combination. An electrode or transducer used to deliver this energy can be used alone or with other electrodes or transducers in a multi-electrode or multi-transducer array. Furthermore, the energy can be applied from within the body (e.g., within the vasculature or other body lumens in a catheter-based approach) and/or from outside the body (e.g., via an applicator positioned outside the body). Furthermore, energy can be used to reduce damage to non-targeted tissue when targeted tissue adjacent to the non-targeted tissue is subjected to neuromodulating cooling.

Neuromodulation using focused ultrasound energy (e.g., high-intensity focused ultrasound energy) can be beneficial relative to neuromodulation using other treatment modalities. Focused ultrasound is an example of a transducer-based treatment modality that can be delivered from outside the body. Focused ultrasound treatment can be performed in close association with imaging (e.g., magnetic resonance, computed tomography, fluoroscopy, ultrasound (e.g., intravascular or intraluminal), optical coherence tomography, or another suitable imaging modality). For example, imaging can be used to identify an anatomical position of a treatment location (e.g., as a set of coordinates relative to a reference point). The coordinates can then entered into a focused ultrasound device configured to change the power, angle, phase, or other suitable parameters to generate an ultrasound focal zone at the location corresponding to the coordinates. The focal zone can be small enough to localize therapeutically-effective heating at the treatment location while partially or fully avoiding potentially harmful disruption of nearby structures. To generate the focal zone, the ultrasound device can be configured to pass ultrasound energy through a lens, and/or the ultrasound energy can be generated by a curved transducer or by multiple transducers in a phased array (curved or straight).

Heating effects of electrode-based or transducer-based treatment can include ablation and/or non-ablative alteration or damage (e.g., via sustained heating and/or resistive heating). For example, a treatment procedure can include raising the temperature of target neural fibers to a target temperature above a first threshold to achieve non-ablative alteration, or above a second, higher threshold to achieve ablation. The target temperature can be higher than about body temperature (e.g., about 37° C.) but less than about 45° C. for non-ablative alteration, and the target temperature can be higher than about 45° C. for ablation. Heating tissue to a temperature between about body temperature and about 45° C. can induce non-ablative alteration, for example, via moderate heating of target neural fibers or of vascular or luminal structures that perfuse the target neural fibers. In cases where vascular structures are affected, the target neural fibers can be denied perfusion resulting in necrosis of the neural tissue. Heating tissue to a target temperature higher than about 45° C. (e.g., higher than about 60° C.) can induce ablation, for example, via substantial heating of target neural fibers or of vascular or luminal structures that perfuse the target fibers. In some patients, it can be desirable to heat tissue to temperatures that are sufficient to ablate the target neural fibers or the vascular or luminal structures, but that are less than about 90° C. (e.g., less than about 85° C., less than about 80° C., or less than about 75° C.).

Renal neuromodulation can include a chemical-based treatment modality alone or in combination with another treatment modality. Neuromodulation using chemical-based treatment can include delivering one or more chemicals (e.g., drugs or other agents) to tissue at a treatment location in a manner that modulates neural function. The chemical, for example, can be selected to affect the treatment location generally or to selectively affect some structures at the treatment location over other structures. The chemical, for example, can be guanethidine, ethanol, phenol, a neurotoxin, or another suitable agent selected to alter, damage, or disrupt nerves. A variety of suitable techniques can be used to deliver chemicals to tissue at a treatment location. For example, chemicals can be delivered via one or more needles originating outside the body or within the vasculature or other body lumens. In an intravascular example, a catheter can be used to intravascularly position a therapeutic element including a plurality of needles (e.g., micro-needles) that can be retracted or otherwise blocked prior to deployment. In other embodiments, a chemical can be introduced into tissue at a treatment location via simple diffusion through a vessel wall, electrophoresis, or another suitable mechanism. Similar techniques can be used to introduce chemicals that are not configured to cause neuromodulation, but rather to facilitate neuromodulation via another treatment modality.

### CONCLUSION

This disclosure is not intended to be exhaustive or to limit the present technology to the precise forms disclosed herein. Although specific embodiments are disclosed herein for illustrative purposes, various equivalent modifications are possible without deviating from the present technology, as those of ordinary skill in the relevant art will recognize. In some cases, well-known structures and functions have not been shown and/or described in detail to avoid unnecessarily obscuring the description of the embodiments of the present technology. Although steps of methods may be presented herein in a particular order, in alternative embodiments the steps may have another suitable order. Similarly, certain aspects of the present technology disclosed in the context of particular embodiments can be combined or eliminated in other embodiments. Furthermore, while advantages associated with certain embodiments may have been disclosed in the context of those embodiments, other embodiments can also exhibit such advantages, and not all embodiments need necessarily exhibit such advantages or other advantages disclosed herein to fall within the scope of the present technology. Accordingly, this disclosure and associated technology can encompass other embodiments not expressly shown and/or described herein.

Certain aspects of the present technology may take the form of computer-executable instructions, including routines executed by a controller or other data processor. In some embodiments, a controller or other data processor is specifically programmed, configured, and/or constructed to perform one or more of these computer-executable instructions. Furthermore, some aspects of the present technology may take the form of data (e.g., non-transitory data) stored or distributed on computer-readable media, including magnetic or optically readable and/or removable computer discs as well as media distributed electronically over networks. Accordingly, data structures and transmissions of data particular to aspects of the present technology are encompassed within the scope of the present technology. The present technology also encompasses methods of both programming computer-readable media to perform particular steps and executing the steps.

The methods disclosed herein include and encompass, in addition to methods of practicing the present technology (e.g., methods of making and using the disclosed devices and systems), methods of instructing others to practice the present technology. For example, a method in accordance with a particular embodiment includes locating a distal end portion of an elongate shaft within or otherwise proximate to a body lumen of a human patient, separating a neuromodulation unit from the distal end portion, expanding a support structure of the neuromodulation unit radially outward relative to a longitudinal axis of the support structure so as to move a therapeutic element carried by the support structure toward a wall of the body lumen, modulating one or more nerves of the patient using the therapeutic element while the neuromodulation unit is separated from the distal end portion, conveying energy toward the therapeutic element via a flexible tether extending between the distal end portion and the neuromodulation unit while modulating the one or more nerves. A method in accordance with another embodiment includes instructing such a method.

Throughout this disclosure, the singular terms “a,” “an,” and “the” include plural referents unless the context clearly indicates otherwise. Similarly, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the terms “comprising” and the like are used throughout this disclosure to mean including at least the recited feature(s) such that any greater number of the same feature(s) and/or one or more additional types of features are not precluded. Directional terms, such as “upper,” “lower,” “front,” “back,” “vertical,” and “horizontal,” may be used herein to express and clarify the relationship between various elements. It should be understood that such terms do not denote absolute orientation. Reference herein to “one embodiment,” “an embodiment,” or similar formulations means that a particular feature, structure, operation, or characteristic described in connection with the embodiment can be included in at least one embodiment of the present technology. Thus, the appearances of such phrases or formulations herein are not necessarily all referring to the same embodiment. Furthermore, various particular features, structures, operations, or characteristics may be combined in any suitable manner in one or more embodiments.

I/We claim:

1. A catheter having a delivery state and a deployed state and comprising:
an elongate shaft having a distal end portion, the shaft being configured to locate the distal end portion within or otherwise proximate to a renal artery of a human patient;
a neuromodulation unit configured to modulate one or more nerves within tissue at or otherwise proximate to a wall of the renal artery, the neuromodulation unit including a therapeutic element and a support structure carrying the therapeutic element, the neuromodulation unit having a collapsed form and an expanded form that resiliently urges the therapeutic element radially outward relative to a longitudinal axis of the neuromodulation unit, the neuromodulation unit being in the collapsed form when the catheter is in the delivery state; and
a tether secured to the neuromodulation unit and operationally associated with the shaft, the tether being sufficiently flexible to allow the neuromodulation unit to move independently of the distal end portion of the shaft when the catheter is in the deployed state and the neuromodulation unit is within the renal artery.

2. The catheter of claim 1, further comprising a junction between the distal end portion of the shaft and the neuromodulation unit, wherein:
the junction is closed when the catheter is in the delivery state;
the junction is open when the catheter is in the deployed state;
the catheter has an intermediate state in which the junction is closed and the neuromodulation unit is in the expanded form; and
the catheter is configured to be in the intermediate state after being in the delivery state and before being in the deployed state.

3. The catheter of claim 1 wherein the neuromodulation unit is configured to at least partially self-align within the renal artery by moving proximally along a longitudinal axis of the renal artery in response to tension on the tether when the catheter is in the deployed state.

4. The catheter of claim 1 wherein:
the therapeutic element includes an electrode;
the catheter further comprises an electrical lead operably connected to the electrode; and
the tether carries the electrical lead between the distal end portion of the shaft and the neuromodulation unit when the catheter is in the deployed state and the neuromodulation unit is within the renal artery.

5. The catheter of claim 1 wherein:
the neuromodulation unit is rotationally interlocking with the distal end portion of the shaft when the catheter is in the delivery state; and
the neuromodulation unit is independent of rotation of the distal end portion of the shaft about an axis of the distal end portion of the shaft when the catheter is in the deployed state and the neuromodulation unit is within the renal artery.

6. The catheter of claim 1 wherein the support structure is helical when the catheter is in the deployed state and the neuromodulation unit is within the renal artery.

7. The catheter of claim 1 wherein:
the neuromodulation unit includes a proximal hub secured to the tether; and
the distal end portion of the shaft and the proximal hub are configured to form a rotationally interlocking junction when the catheter is in the delivery state.

8. The catheter of claim 1 wherein the tether is secured to the shaft.

9. The catheter of claim 1 wherein the tether extends through the distal end portion of the shaft.

10. The catheter of claim 1 wherein:
the neuromodulation unit includes a proximal hub secured to the tether;
the proximal hub includes a proximal pin;
the distal end portion of the shaft includes a bore; and
the distal end portion of the shaft and the proximal hub are configured to form a mating junction when the catheter is in the delivery state, the proximal pin being at least partially received within the bore at the mating junction.

11. The catheter of claim 10 wherein:
the tether is secured to and/or extends through the proximal pin; and
at least a portion of the proximal pin is tapered with decreasing width in a direction away from the support structure.

12. The catheter of claim 10 wherein:
the proximal pin includes—
two or more first locking features circumferentially spaced apart around an outer surface of the proximal pin, and
two or more first intervening regions individually positioned between circumferentially adjacent first locking features;
the bore includes—
two or more second locking features circumferentially spaced apart around an inner surface of the bore, and
two or more second intervening regions individually positioned between circumferentially adjacent second locking features;
the first locking features are configured to move longitudinally through the second intervening regions as the proximal pin moves into the bore in response to tension on the tether;
the second locking features are configured to move longitudinally through the first intervening regions as the proximal pin moves into the bore in response to tension on the tether; and
the distal end portion of the shaft includes a resilient member operably positioned within the bore, the resilient member being configured to resiliently deform as the proximal pin moves into the bore in response to tension on the tether, the resilient member being configured to resiliently urge the first locking features into operable engagement with the second locking features when tension on the tether is at least partially released.

13. The catheter of claim 12 wherein:
the individual first locking features include a first distal edge portion and a first proximal edge portion;
the individual second locking features include a second distal edge portion and a second proximal edge portion, the second distal edge portion including one or more first notches;
the bore includes a ledge having two or more second notches;
the first proximal edge portions are configured to interact with the second distal edge portions to cause the proximal pin to rotate relative to the bore so as to move the first locking features into alignment with the second intervening regions as the proximal pin moves into the bore in response to tension on the tether;
the first proximal edge portions are configured to interact with the second notches to cause the proximal pin to rotate relative to the bore so as to move the first locking features from being aligned with the second intervening regions toward being out of alignment with the second intervening regions as the proximal pin moves into the bore in response to tension on the tether; and
the first distal edge portions are configured to interlock with the first notches as the resilient member urges the first locking features into operable engagement with the second locking features when tension on the tether is at least partially released.

14. The catheter of claim 13 wherein:
the proximal pin is configured to move into the bore in response to a first period of tension on the tether; and
the first proximal edge portions are configured to interact with the second notches to cause the proximal pin to rotate relative to the bore so as to move the first locking features from being out of alignment with the second intervening regions toward being aligned with the second intervening regions during a second period of tension on the tether, the second period being temporally spaced apart from and following the first period.

15. The catheter of claim 1 wherein:
the therapeutic element is a first therapeutic element;
the neuromodulation unit includes a second therapeutic element; and
the support structure includes—
a proximal hub secured to the tether,
a first arm extending distally from the proximal hub, the first arm carrying the first therapeutic element and being configured to resiliently urge the first therapeutic element radially outward relative to the longitudinal axis of the support structure in a first radial direction when the catheter is in the deployed state and the neuromodulation unit is within the renal artery, and
a second arm extending distally from the proximal hub, the second arm carrying the second therapeutic element and being configured to resiliently urge the second therapeutic element radially outward relative to the longitudinal axis of the support structure in a second radial direction when the catheter is in the deployed state and the neuromodulation unit is within the renal artery, the second radial direction being circumferentially spaced apart from the first radial direction around the longitudinal axis of the support structure.

16. The catheter of claim 15 wherein:
the distal end portion of the shaft includes a distal rim;
the tether extends through the distal rim; and
the proximal hub caps the distal rim when the catheter is in the delivery state.

17. The catheter of claim 15 wherein:
the first therapeutic element is at least proximate to a distal tip of the first arm; and
the second therapeutic element is at least proximate to a distal tip of the second arm.

18. The catheter of claim 15, further comprising a distal hub, wherein the first and second arms individually extend from the proximal hub to the distal hub.

19. The catheter of claim 18 wherein:
the first therapeutic element is configured to contact a first segment of the wall of the renal artery when the catheter is in the deployed state and the neuromodulation unit is within the renal artery;
the second therapeutic element is configured to contact a second segment of the wall of the renal artery when the catheter is in the deployed state and the neuromodulation unit is within the renal artery; and
the first and second segments are different.

20. A catheter, comprising:
an elongate shaft having a distal end portion, the shaft being configured to locate the distal end portion within or otherwise proximate to a body lumen of a human patient;
a neuromodulation unit configured to modulate one or more nerves within tissue at or otherwise proximate to a wall of the body lumen, the catheter having a delivery state in which the neuromodulation unit is translationally coupled to the distal end portion of the shaft and a deployed state in which the neuromodulation unit is translationally decoupled from the distal end portion of the shaft, the neuromodulation unit including a support structure and a therapeutic element operably coupled to the support structure, the support structure being configured to resiliently urge the therapeutic element radially outward relative to a longitudinal axis of the neuromodulation unit when the catheter is in the deployed state and the neuromodulation unit is within the body lumen; and
a tether secured to the neuromodulation unit and operationally associated with the distal end portion of the shaft, the tether being configured to restrict a separation distance between the distal end portion of the shaft and the neuromodulation unit when the catheter is in the deployed state and the neuromodulation unit is within the body lumen.

21. A method, comprising:
locating a distal end portion of an elongate shaft within or otherwise proximate to a body lumen of a human patient;
separating a neuromodulation unit from the distal end portion of the shaft while maintaining a connection therebetween via a flexible tether;
expanding a support structure of the neuromodulation unit radially outward relative to a longitudinal axis of the support structure so as to move a therapeutic element carried by the support structure toward a wall of the body lumen; and
modulating one or more nerves of the patient using the therapeutic element while the neuromodulation unit is separate from the distal end portion of the shaft by transmitting energy to the therapeutic element via the flexible tether.

22. The method of claim 21, further comprising accommodating, via the tether, relative movement between the neuromodulation unit and the distal end portion of the shaft due to the patient's respiration while modulating the one or more nerves.

23. The method of claim 21 wherein accommodating the relative movement includes accommodating the relative movement while the neuromodulation unit is within a renal artery of the patient, the distal end portion of the shaft is within an aorta of the patient, and the tether extends through a renal ostium of the patient.

24. The method of claim 21 wherein expanding the neuromodulation unit includes expanding the neuromodulation unit into a helical shape.

25. The method of claim 21 wherein separating the neuromodulation unit from the distal end portion of the shaft includes:
applying tension to the neuromodulation unit via the tether to unlock the neuromodulation unit from the distal end portion of the shaft; and
releasing the tension to allow a resilient member of the distal end portion of the shaft to urge the neuromodulation unit distally outward relative to the distal end portion of the shaft.

26. The method of claim 21 further comprising using the tether to restrict a separation distance between the distal end portion of the shaft and the neuromodulation unit while modulating the one or more nerves.

27. The method of claim 21, further comprising at least partially aligning the neuromodulation unit within the body lumen by moving the neuromodulation unit proximally along a longitudinal axis of the body lumen in response to tension on the tether.

28. The method of claim 27 wherein at least partially aligning the neuromodulation unit includes applying tension to the tether while a distalmost contact point between the tether and the distal end portion of the shaft is aligned with a longitudinal axis of the body lumen.
